SEQUENCE LISTING (6 sequences)

SEQ ID NO: 1 - DNA, 24 bp, synthetic construct
  misc_feature 1..24: note = primer
  source 1..24: mol_type = other DNA
gccagctgacaacttgattgggtt

SEQ ID NO: 2 - DNA, 20 bp, synthetic construct
  misc_feature 1..20: note = primer
  source 1..20: mol_type = other DNA
ggcgcttcaaacctgaagga

SEQ ID NO: 3 - RNA, 19 nt, Homo sapiens
  source 1..19: mol_type = unassigned RNA
gagacagttgatagccaaa

SEQ ID NO: 4 - RNA, 21 nt, Homo sapiens
  source 1..21: mol_type = unassigned RNA
aagcatgcaccactacactca

SEQ ID NO: 5 - DNA, 21 bp, synthetic construct
  source 1..21: mol_type = other DNA
tgagtgtagtggtgcatgctt

SEQ ID NO: 6 - DNA, 21 bp, synthetic construct
  misc_feature 1..5: note = RNA - locked nucleic acids
  misc_feature 6..16: note = DNA
  misc_feature 17..21: note = RNA - locked nucleic acids
  source 1..21: mol_type = other DNA
  modified_base 1..21: mod_base = OTHER; note = Phosphodiester bonds are replaced by phosphothioate linkage
tgagtgtagtggtgcatgctt

(19) United States
(12) Patent Application Publication
(10) Pub. No.: US 2025/0313833 A1
(43) Pub. Date: Oct. 9, 2025

(54) ANTISENSE OLIGONUCLEOTIDE FOR TREATING MELANOMA

(71) Applicant: Katholieke Universiteit Leuven, Leuven (BE)

(72) Inventors: Eleonora Leucci, Leuven (BE); Sonia Cinque, Leuven (BE)

(73) Assignee: Katholieke Universiteit Leuven, Leuven (BE)

(21) Appl. No.: 18/865,456

(22) PCT Filed: May 19, 2023
(86) PCT No.: PCT/EP2023/063472

§ 371 Date: Nov. 13, 2024

(30) Foreign Application Priority Data
May 20, 2022 (EP) 22174555.7
Oct. 13, 2022 (EP) 22201236.1

(51) Int. Cl. C12N 15/113; A61P 35/00

(52) U.S. Cl. CPC C12N 15/113; A61P 35/00; C12N 2310/11; C12N 2310/14

(57) ABSTRACT

The present invention relates to an oligonucleotide for treating melanoma, in particular an inhibitor of a long intergenic non-protein coding RNA to decrease activation of the Integrated Stress Response in melanoma cells. More specifically, the present invention is directed to an inhibitor of LINC00941 expression for use in the treatment of melanoma in a subject.

### FIELD OF THE INVENTION

The present invention relates to an oligonucleotide for treating melanoma, in particular an inhibitor of a long intergenic non-protein coding RNA to decrease activation of the Integrated Stress Response in melanoma cells. More specifically, the present invention is directed to the use of an inhibitor of LINC00941 expression in the treatment of melanoma.

### BACKGROUND OF THE INVENTION

Despite the recent advances in immunotherapy, melanoma is still the leading cause of skin cancer. Overall survival upon treatment with a combination of anti-PD1 and anti-CTLA4 is barely 52% (Larkin et al., 2019). In tumor resistant to immune checkpoint inhibition but with similar mutation burdens, three scenarios have been recognized based on the presence of T-cells within the tumor: 1. Immune deserts, where T cells are absent from the tumor and its periphery; 2. Immune excluded, where the T cells are all around the tumor, but do not infiltrate it and 3. Inflamed tumors, where T-cells are in and around the tumor but still not sufficient to efficiently attack it. Therefore, it was an object of the present invention to increase T-cell mediated killing leading to a more efficient alternative treatment against melanoma.

The inventors of the present invention have found that the knock-down of the long intergenic non-protein-coding RNA LINC00941 decreases activation of the Integrated Stress Response (ISR) in melanoma cells. More specifically, it was unexpectedly found that knock-down of LINC00941 increases T-cell mediated killing of melanoma cells. Moreover, it was found that expression levels of LINC00941 is higher in melanoma cells with invasive/therapy resistant signature, is higher in patients showing resistance to immunotherapy, and is retained in polysomes upon acquisition of resistance to targeted therapy.

### SUMMARY OF THE INVENTION

The present invention relates to an inhibitor of LINC00941 expression for use in the treatment of melanoma in a subject.

In one embodiment, the inhibitor of LINC00941 expression for use according to the invention is for use in combination with an immune checkpoint inhibitor and/or a therapeutic agent for targeted therapy.

In one embodiment, the subject has, or is at risk of having, melanoma resistant to an immune checkpoint inhibitor and/or to a therapeutic agent for targeted therapy.

In one embodiment, the melanoma is, or is at risk of being, a melanoma resistant to an immune checkpoint inhibitor and/or to a therapeutic agent for targeted therapy.

In one embodiment, the immune checkpoint inhibitor is selected from the group consisting of PD-1 inhibitors, CTLA-4 inhibitors, PD-L1 inhibitors and LAG3 inhibitors.

In one embodiment, the therapeutic agent for targeted therapy is selected from the group consisting of B-Raf inhibitors and MEK-inhibitors.

In one embodiment, melanoma is invasive melanoma and/or metastatic melanoma.

In one embodiment, the inhibitor of LINC00941 expression for use according to the invention is selected from the group consisting of silencing RNA (siRNA) and antisense oligonucleotide (ASO).

In one embodiment, the inhibitor of LINC00941 expression for use according to the invention is an ASO targeting LINC00941.

In one embodiment, the inhibitor of LINC00941 expression for use according to the invention, is an ASO inducing degradation of LINC00941, preferably RNAse H-mediated degradation of LINC00941.

In one embodiment, the inhibitor of LINC00941 expression for use according to the invention is an ASO having an overall nucleotide sequence length of at least 10 nucleotides, preferably an ASO having an overall nucleotide sequence length of at least 12, 13, 14, 15, 16, 17, 18, 19 or 20 nucleotides, more preferably an ASO having an overall nucleotide sequence length of at least 21 nucleotides.

In one embodiment, the inhibitor of LINC00941 expression for use according to the invention, is an ASO comprising a contiguous nucleotide sequence that is at least 90%, 92%, 94%, 96%, 97%, 98% 99% or preferably 100%, complementary to LINC00941 and that is of length of nucleotide sequence ranging from 10 to 50 nucleotides, preferably ranging from 10 to 40, 10 to 39, 10 to 38, 10 to 37, 10 to 37, 10 to 36, 10 to 35, 10 to 34, 10 to 33, 10 to 31 or 10 to 30 nucleotides, more preferably ranging from 11 to 29, 12, to 28, 13 to 27, 14 to 26 or 15 to 25 nucleotides.

In one embodiment, the inhibitor of LINC00941 expression for use according to the invention is an ASO comprising modified nucleotides selected from the list comprising: 2′—O—Me, 2′—F, MOE, LNA or a combination thereof.

In one embodiment, the inhibitor of LINC00941 expression for use according to the invention is a gapmer.

In another aspect, the present invention is directed to a method for the treatment and/or prevention of melanoma in a subject in need thereof, said method comprising administering to said subject an inhibitor of LINC00941 expression.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1 is graph showing qPCR expression of LINC00941 in patient-derived xenograft (PDX) model on treatment with vehicle or targeted therapy (DT relapse). DT: Dabrafenib-Trametinib. *p value calculated by t-test is 0.0202.

FIG. 2 is a graph showing qPCR expression of LINC00941 in ribosome profiling-derived fractions of SK-MEL-28 cells in control (ctrl) and after activation of the Integrated Stress Response (ISR activation). LINC00941 co-sediment with the small ribosomal subunit and accumulate in polysomes upon ISR activation.

FIG. 3 is a graph showing the association of LINC00941 in ribosome profiling-derived fractions in 3 drug-resistant models showing active ISR (indicated as MEL058, MEL077 and MEL020—see Vendramin et al. 2021,. Counts normalized on the size of the library and on total RNA are plotted. The accumulation of LINC00941 in the polysome fraction in these models suggests that LINC00941 may reduce translation of mRNAs during the ISR by steric hindrance.

FIG. 4 is a graph showing the differential co-sedimentation with polysomes of the ATF4 mRNA, as detected by RNAseq on MM099 cells upon knock-down of LINC00941 (siLINC00941) or in control (siCtrl). This result suggests that knock-down of LINC00941 regulates ATF4 expression at a translational level.

FIG. 5 is a western blot showing level of Vinculin, MITF, ATF4, phosphorylated EIF2 α (P-eIF2 α) and eIF2 α in MM099 cells treated with a siRNA against LINC00941 (siLINC00941) or with a control siRNA (siCTRL). This result confirms the decrease in ISR-induced ATF4 expression upon knock down of LINC00941.

FIG. 6 is a graph showing the expression level of LINC00941 in primary and metastatic sample in the melanoma TCGA cohort. *p value in unpaired two-tailed t-test was below 0.05.

FIG. 7 is a graph showing the correlation of LINC00941 expression with the proliferative (PRO) and invasive (INV) signature in the melanoma TCGA cohort. ****p unpaired two-tailed t-test was below 0.0001.

FIG. 8 is the analysis of the TCGA melanoma cohort. The graph in panel A shows the copy number alteration of the LINC00941 locus. The graph in panel B is a Kaplan-Meyer curve showing correlation of LINC00941 copy number with patients' survival. *p value calculated by long-rank (Mantel-Cox) test.

FIG. 9 is the quantification of a co-culturing experiment between melanoma cells and HLA-matched peripheral blood mononuclear cells (PBMCs) upon treatment with a siRNA against LINC00941 (siLINC00941) or with a control siRNA (siCTRL) or not transfected controls (MOCK). Panel A shows MM099 confluency. Panel B shows PBMCs confluency. This experiment shows activation of PBMCs proliferation and increase in MM099 melanoma cell killing upon LINC00941 silencing. *p value (in both instances) calculated by two-ways ANOVA for the interaction between time and treatment was below 0.05.

FIG. 10 is the quantification of an experiment wherein PBMCs were cultured in medium derived from LINC00941 (siLINC00941) or control siRNA (siCTRL) transfected melanoma cells. This experiment shows that PBMCs proliferation seen upon LINC00941 knock-down in FIG. 9B is activated by a direct contact between melanoma cells and the immune cells.

FIG. 11 Panel A is graph showing qPCR expression of LINC00941 in the polysome fractions of humanized PDX model MEL006, partially responding to anti-PD1 (Vendramin et al.,2021), in control condition (vehicle) or after treatment with anti-PD1 (a-PD1; nivolumab). p value as calculated by unpaired t test is 0.0939. Panel B is a graph showing the qPCR expression of LINC00941 in humanized PDX models treated with Nivolumab (Vendramin et al. 2021) in responders and non-responders. The Expression was normalized on the average expression of the respective vehicle treated cohort ***:p value as calculated by unpaired t test is inferior to 0.0001.

FIG. 12 Panel A is a graph showing LINC00941 expression the immunotherapy treated cohort from Hugo et al. 2016.

FIG. 13 is a graph showing successful knock-down of LINC00941 using an antisense oligonucleotide overlapping the sequence of siRNA#3. **:p value as calculated by paired two-tailed test was 0.0020.

FIG. 14 is a Kaplan-Meyer curve showing the effect of systemic inhibition of LINC00941 in immune-compromised (NOG) and immune-competent (hu-NOG) melanoma PDX model resistant to Immune checkpoint inhibition. *:p value as calculated by long-rank (Mantel-Cox) test was 0.0130. LISR: LINC00941.

FIG. 15 shows tumor growth over days of a humanized melanoma PDX model resistant to Immune checkpoint inhibition upon systemic inhibition of LINC00941 alone or in combination with immune checkpoint inhibitors. p values were calculated by one-way ANOVA. **p value<0.001; ****p value<0.0001. LISR: LINC00941.

### DETAILED DESCRIPTION OF THE INVENTION

Here the inventors have found that the knock-down (KD) of the long intergenic non-protein-coding RNA LINC00941 decreases activation of the Integrated Stress Response in melanoma cells, which is involved in targeted therapy and immunotherapy resistance. Accordingly, the inventors have found that KD of LINC00941 increases T-cell mediated killing of melanoma cells. Expression of LINC00941 was furthermore found by the inventors to be higher in melanoma cells with invasive/therapy resistant signature, to be retained at polysomes upon acquisition of resistance to targeted therapy and to be higher in patients showing resistance to immunotherapy.

The present invention hence relates to an inhibitor of LINC00941 expression for use in the treatment of melanoma.

In one embodiment, the inhibitor of LINC00941 expression of the invention is selected from the group consisting of silencing RNA (siRNA) and antisense oligonucleotide (ASO). In one embodiment, the inhibitor of LINC00941 expression of the invention is a siRNA, preferably a siRNA targeting LINC00941. In one embodiment, the inhibitor of LINC00941 expression of the invention is an antisense oligonucleotide (ASO), preferably an ASO targeting LINC00941.

The present invention hence also relates to an antisense oligonucleotide (ASO) targeting, and/or degrading, and/or inhibiting the expression of, LINC00941 for use in the treatment of melanoma.

The term “targeting” is used herein in reference to a compound (the inhibitor of LINC00941 expression in the context of the invention) leading to the degradation of another compound (LINC00941 in the context of the invention). As defined herewith the term “degradation” represents the degradation of LINC00941 caused by the siRNA or ASO according to the invention. The degradation can be complete, meaning that 100% of LINC00941 is degraded, or partial meaning that at least about 40%, at least about 50%, at least about 60%, at least about 70%, at least about 80%, at least about 90%, or at least about 95% of LINC00941 is degraded.

The terms “antisense oligonucleotide” or “ASO” are used herein interchangeably in reference to a modified single stranded antisense oligonucleotide comprising at least one region complementary to its target RNA, LINC00941 in the context of the invention, and that upon binding to said target RNA, trigger the degradation of said target RNA.

The term “small interfering RNA” or “siRNA” are used herein interchangeably in reference to a class of double-stranded RNA operating within the RNA interference (RNAi) pathway. siRNAs interfere with the expression of specific genes at post-transcriptional level by binding to an enzyme (RISC) that will catalyze the cleavage of both the siRNA and target mRNA. RNAi can be initiated by double-stranded RNA molecules (double stranded RNA or short hairpin RNA) that, when introduced into a cell are cleaved by Dicer into a mixture of double stranded siRNA. In mammalian cells, the siRNAs that are naturally produced by Dicer are typically 21-23 bp in length, with a 19 or 20 nucleotides duplex sequence, two-nucleotide 3′ overhangs and 5′-triphosphate extremities.

Unless specified otherwise, the term “LINC00941” is used herein in reference to all isoforms, or splice variants, of the long intergenic non-protein coding RNA 941 produced by the gene of NCBI Entrez geneID reference 100287314, of Ensembl references ENSG00000235884 and ENSG00000285517, and of HUGO Gene Nomenclature Committee reference HGNC: 48635. The long intergenic non-protein coding RNA 941 gene of references may also referred to in the literature as LncRNA ISR Regulator (LISRR).

In one embodiment, LINC00941 is all isoforms, or splice variants, of the long intergenic non-protein coding RNA 941 produced by the gene of NCBI Entrez geneID reference 100287314, of Ensembl references ENSG00000235884 and ENSG00000285517, and of HUGO Gene Nomenclature Committee reference HGNC:48635 wherein said isoforms or splice variants comprise a sequence that is at least and/or about 90%, 91%, 92%, 93%, 94%, 95%, 96%, 97%, 98%, 99% or 100% identical any one of SEQ ID NO. 3 and SEQ ID NO. 4

In one embodiment, LINC00941 is the isoforms, or splice variants, of the long intergenic non-protein coding RNA 941, of Ensembl reference ENST00000648050.1, ENST00000650198.1, ENST00000650193.1, ENST00000649555.1, ENST00000649390.1, ENST00000649354.1, ENST00000649324.1, ENST00000649043.2, ENST00000648700.1, ENST00000648367.1, ENST00000550292.2, ENST00000547804.2 and ENST00000650286.1.

In one embodiment, LINC00941 is all isoforms, or splice variants, of the long intergenic non-protein coding RNA 941 produced by the gene of Ensembl reference ENSG00000285517.

In one embodiment, LINC00941 is all isoforms, or splice variants, of the long intergenic non-protein coding RNA 941 produced by the gene of Ensembl reference ENSG00000285517, wherein said isoforms or splice variants comprise a sequence that is at least and/or about 90%, 91%, 92%, 93%, 94%, 95%, 96%, 97%, 98%, 99% or 100% identical to any one of SEQ ID NO. 3 and SEQ ID NO. 4.

In one embodiment, LINC00941 is the isoforms, or splice variants, of the long intergenic non-protein coding RNA 941, of Ensembl reference ENST00000648050.1, ENST00000650198.1, ENST00000650193.1, ENST00000649555.1, ENST00000649390.1, ENST00000649354.1, ENST00000649324.1, ENST00000649043.2, ENST00000648700.1, ENST00000648367.1, ENST00000550292.2 and ENST00000547804.2.

In one embodiment, LINC00941 is the isoform, or splice variant, of the long intergenic non-protein coding RNA 941 of Ensembl reference ENST00000648050.1 produced by the gene of Ensembl reference ENSG00000285517.

The present invention relates to a siRNA or an ASO targeting LINC00941, and/or degrading LINC00941 and/or inhibiting the expression of LINC00941.

In one embodiment, the siRNA or ASO of the invention has an overall nucleotide sequence length of at least 10 nucleotides, preferably of at least 12, 13, 14, 15, 16, 17, 18, 19 or 20 nucleotides, more preferably of at least 21 nucleotides.

In one embodiment, the siRNA or ASO of the invention has an overall nucleotide sequence length inferior or equal to 50 nucleotides, preferably inferior or equal to 40, 39 38, 37, 36, 35, 34, 33, 32, or 31 nucleotides, more preferably inferior or equal to 30, 29, 28, 27, 26, 25, 24, 23, 22 or 21 nucleotides. In the context of the present invention, the term ‘inferior to’ is to be understood as less or lesser than.

In one embodiment, the siRNA or ASO of the invention has an overall nucleotide sequence length ranging from 10 to 50 nucleotides, preferably ranging from 10 to 40, 10 to 39, 10 to 38, 10 to 37, 10 to 36, 10 to 35, 10 to 34, 10 to 33, 10 to 31 or 10 to 30 nucleotides, more preferably ranging from 11 to 29, 12, to 28, 13 to 27, 14 to 26 or 15 to 25 nucleotides.

In one embodiment, the siRNA or ASO of the invention has an overall nucleotide sequence length of 15, 16, 17, 18, 19, 20, 21, 22, 23, 24 or 25 nucleotides.

The siRNA or ASO of the invention comprises a nucleotide sequence, preferably a contiguous nucleotide sequence, that is complementary to LINC00941.

In the context of the present invention, the term “complementary” is to be understood as a sequence capable of hybridizing specifically with another given (target) sequence. Hybridization refers to the pairing or annealing to a target, herein under physiological conditions, typically via hydrogen bonding between complementary nucleotides, such as Watson-Crick, Hoogsteen or reversed Hoogsteen hydrogen bonding. Hybridizing specifically means that the pairing or annealing is specific to the target (no off-targets effects, or at least no substantial off-targets effects). While preferred, full complementarity is not necessarily required, mismatches may be tolerated to some extent, provided there is sufficient complementarity to cause hybridisation. The conditions appropriate for hybridization between two nucleic acids depend on the length of the nucleic acids and the degree of complementation, variables well known in the art. Typically, the length for a hybridisable nucleic acid is at least about 10 nucleotides. Illustrative minimum lengths for a hybridisable nucleic acid are: at least about 15 nucleotides; at least about 20 nucleotides; at least about 22 nucleotides; at least about 25 nucleotides; and at least about 30 nucleotides.

In some embodiments, the siRNA or ASO of the invention is at least about 60% complementary to one of its target RNA LINC00941 sequences over a stretch of at least 8, such as at least 10, 12, 14, 16, 18, 20, 22, 24 contiguous nucleotides. For example, the siRNA or ASO of the invention is at least about 65% complementary, at least about 70% complementary, at least about 75% complementary, at least about 80% complementary, at least about 85% complementary, at least about 90% complementary, at least about 95% complementary, at least about 98% complementary, at least about 99% complementary or 100% complementary to one of the target RNA LINC00941 sequences over a stretch of at least 8 such as at least 10, 12, 14, 16, 18, 20, 22, 24 contiguous nucleotides.

In one embodiment, the siRNA or ASO of the invention comprises a nucleotide sequence, preferably a contiguous nucleotide sequence complementary to LINC00941 that is at least 10 nucleotides in length of nucleotide sequence, preferably at least 12, 13, 14, 15, 16, 17, 19 or 20 nucleotides in length of nucleotide sequence, more preferably of at least 21 nucleotides in length of nucleotide sequence.

In one embodiment, the siRNA or ASO of the invention comprises a nucleotide sequence, preferably a contiguous nucleotide sequence complementary to LINC00941 that is at most 50 nucleotides in length of nucleotide sequence, preferably at most 40, 39, 38, 37, 36, 35, 34, 33, 32, or 31 nucleotides in length of nucleotide sequence, more preferably at most 30, 29, 28, 27, 26, 25, 24, 23, 22 or 21 nucleotides in length of nucleotide sequence.

In one embodiment, the siRNA or ASO of the invention comprises a nucleotide sequence, preferably a contiguous nucleotide sequence complementary to LINC00941 of length of nucleotide sequence ranging from 10 to 50 nucleotides, preferably ranging from 10 to 40, 10 to 39, 10 to 38, 10 to 37, 10 to 37, 10 to 36, 10 to 35, 10 to 34, 10 to 33, 10 to 31 or 10 to 30 nucleotides, more preferably of length of nucleotide sequence ranging from 11 to 29, 12, to 28, 13 to 27, 14 to 26 or 15 to 25 nucleotides.

While preferred, perfect complementarity is not necessary. In one embodiment, the contiguous nucleotide sequence complementary to LINC00941 is complementary to LINC00941 over its entire length. In one embodiment, the contiguous nucleotide sequence complementary to LINC00941 comprises 1, 2, 3, 4, 5 or more mismatches.

In one embodiment, the siRNA or ASO of the invention comprises, or consists of, a contiguous nucleotide sequence that is at least 90%, 92%, 94%, 96%, 97%, 98% 99% or preferably 100%, complementary to LINC00941 and that is of length of nucleotide sequence ranging from 10 to 50 nucleotides, preferably ranging from 10 to 40, 10 to 39, 10 to 38, 10 to 37, 10 to 37, 10 to 36, 10 to 35, 10 to 34, 10 to 33, 10 to 31 or 10 to 30 nucleotides, more preferably of length ranging from 11 to 29, 12, to 28, 13 to 27, 14 to 26 or 15 to 25 nucleotides.

In one embodiment, the ASO of the invention comprises ribonucleotides and/or deoxyribonucleotides.

In one embodiment, the ASO of the invention induces RNAse H-mediated degradation of LINC00941. In one embodiment, the ASO of the invention comprises a region comprising deoxyribonucleotides that is complementary to LINC00941.

The siRNA or ASO of the invention may comprise one or more modifications aiming at improving stability, potency and/or selectivity of said siRNA or ASO.

In one embodiment, the ASO of the invention comprises phosphothioate bonds in place of phosphodiester bonds. In one embodiment, all phosphodiester bonds within the ASO of the invention are replaced by phosphothioate bonds.

In one embodiment, the ASO of the invention comprises modified nucleotides selected from the list comprising, or consisting of, 2′—O—Me, 2′—F, MOE, LNA or a combination thereof. In one embodiment, the ASO of the invention comprises LNA modified nucleotides.

“2′—O—Me” refers to a nucleotide modification consisting of the addition on ribose of a 2′—O-methyl group. “2′—F” refers to a nucleotide modification consisting of the addition on ribose of a 2′-fluoro group. “MOE” refers to a nucleotide modification consisting of the addition on ribose of a 2′—O-methoxyethyl group. “LNA” refers to Locked Nucleic Acid, a nucleotide modification consisting of the introduction of a bridge between the 2′ oxygen and 4′ carbon of the ribose.

In one embodiment, the ASO of the invention comprises at least 1, 2, 3, 4, 5, 6, 7, 8, 9 or 10 LNA modified nucleotides. In one embodiment, the 5′ end of the ASO of the invention consists of 1, 2, 3, 4, 5, 6, 7, 9 or 10 LNA modified nucleotides and/or the 3′ end of the ASO of the invention consists of 1, 2, 3, 4, 5, 6, 7, 9 or 10 LNA modified nucleotides.

In one embodiment, the ASO of the invention targets, or is complementary to, the sequence SEQ ID NO. 4. In one embodiment, the ASO of the invention comprises, or consists of, a nucleic acid of sequence SEQ ID NO. 5. In one embodiment, the ASO of the invention comprises, or consists of, SEQ ID NO. 6. In one embodiment, the ASO of the invention is at least and/or about 90%, 91%, 92%, 93%, 94%, 95%, 96%, 97%, 98%, 99% or 100% complementary to SEQ ID NO. 4.

In one embodiment, the inhibitor of LINC00941 expression of the invention is a gapmer. The term “gapmer” is used herein in reference to an ASO comprising, or consisting of, a central gap region consisting of deoxyribonucleotides and one or two flanking regions comprising, or consisting of, modified ribonucleotides, preferably 2′—O—Me, 2′—F, MOE, LNA modified ribonucleotides or a combination thereof. more preferably comprising LNA modified ribonucleotides.

In one embodiment, the inhibitor of LINC00941 expression for use in the treatment of melanoma of the invention is for use in combination with an immune checkpoint inhibitor and/or for use in combination with a therapeutic agent for targeted therapy.

Examples of immune checkpoint inhibitor that may be used in combination with the inhibitor of LINC00941 expression of the invention include, without being limited to, anti PD-1 antibodies, such as pembrolizumab, pidilizumab and nivolumab, Anti PD-L1 antibodies, such as atezolizumab, avelumab, durvalumab, anti CTLA4 antibodies such as ipilimumab and tremelimumab and anti LAG3 antibodies, such as relatlimab.

In one embodiment, the immune checkpoint inhibitor is selected from the group consisting of PD-1 inhibitors, CTLA-4 inhibitors, PD-L1 inhibitors and LAG3 inhibitors.

In one embodiment, the immune checkpoint inhibitor is selected from the group consisting of anti PD-1 antibody, anti CTLA-4 antibody, anti PD-L1 antibody, and anti LAG3 antibodies.

Examples of an agent for targeted therapy that may be used in combination with the inhibitor of LINC00941 expression of the invention include, without being limited to, B-Raf inhibitors, such as dabrafenib, and MEK inhibitors, such as trametinib.

In one embodiment, the therapeutic agent for targeted therapy is selected from the group consisting of B-Raf inhibitors and MEK inhibitors.

In one embodiment, the therapeutic agent for targeted therapy is selected from the group consisting of dabrafenib and trametinib.

In one embodiment, melanoma is invasive and/or metastatic melanoma.

“Invasive melanoma” refers herein to a melanoma that is not confined to the upper layer of the epidermis.

“Metastatic melanoma” refers herein to melanoma that has metastasized.

In one embodiment melanoma is, or is at risk of being, melanoma resistant to therapy. “melanoma resistant to therapy” refers to herein to melanoma that does not, or partially, respond to said therapy.

Methods to determine the risk of a melanoma being resistant to therapy are known to the skilled artisan and include, without being limited to, the use of biomarkers, such as mutations or expression of specific gene products, indicative of a risk of therapy resistance.

In one embodiment, melanoma is, or is at risk of being, melanoma resistant to immune checkpoint inhibitor and/or to a therapeutic agent for targeted therapy.

Examples of immune checkpoint inhibitors that may be considered in the context of determining the resistance of melanoma include, without being limited to, anti PD-1 antibodies, such as pembrolizumab, pidilizumab and nivolumab, AntiPD-L1 antibodies, such as atezolizumab, avelumab, durvalumab, antiCTLA4 antibodies such as ipilimumab and tremelimumab and anti LAG3 antibodies such as, relatlimab.

Examples of therapeutic agent for targeted therapy that may be considered in the context of determining the resistance of melanoma include, without being limited to, B-Raf inhibitors, such as dabrafenib, and MEK inhibitors, such as trametinib.

In the context of the invention, the subject is human.

In one embodiment, the subject has, or is at risk of having, an invasive melanoma.

In one embodiment, the subject has, or is at risk of having, a metastatic melanoma.

In one embodiment, the subject has, or is at risk of having, a melanoma resistant to therapy. In one embodiment, the subject has, or is at risk of having, a melanoma resistant to immune checkpoint inhibitor and/or to a therapeutic agent for targeted therapy.

In another aspect, the present invention is directed to a method for the treatment and/or prevention of melanoma in a subject in need thereof, said method comprising administering to said subject an inhibitor of LINC00941 expression.

In a particular embodiment, said method for the treatment and/or prevention of melanoma in a subject in need thereof comprises administering to said subject an inhibitor of LINC00941 expression wherein said inhibitor can be selected from a siRNA and/or a ASO comprising a nucleotide sequence, preferably a contiguous nucleotide sequence, that is complementary to LINC00941.

### EXAMPLES

The present invention is further illustrated by the following examples.

### Example 1: Knock Down of LINC00941 Increases the Immunogenicity of Melanoma Cells

### Materials and Methods

### Polysome Profiling

Tumor pieces and SK-MEL-28 cells (15-cm dishes per each condition) were plated to have 70% confluency after 72 h. The following day to induce ISR, cells were treated with 20 μM salubrinal (Sigma-Aldrich). 72 h after the start of the treatment, cells were treated with 100 μg/ml of cycloheximide (Sigma-Aldrich) for 12 min at 37° C., collected, and resuspended in lysis buffer (30 mM Tris-HCl, 150 mM KCl, 10 mM MgCl2, supplemented with 1 mM DTT [Sigma-Aldrich], 100 μg/ml cycloheximide, 20 U/μl SUPERase-IN RNase Inhibitor [Invitrogen, Thermo Fisher Scientific], and Halt Protease and Phosphatase Inhibitor Single-Use Cocktail [Life Technologies] before the start of the experiment). Lysates were then incubated agitating at 4° C. for 35 min, and then centrifuged at 17,000 rcf for 15 min at 4° C. Lysates were loaded on a sucrose gradient (the linear sucrose gradient 5-20% was generated from two different solutions, sucrose 5 and 20%, made with buffer G (20 mM Tris-HCl, 100 mM KCl, 10 mM MgCl₂ supplemented with 1 mM DTT and 100 μg/ml cycloheximide before the start of the experiment). Samples were then centrifugated in an SW41Ti rotor (Beckman Coulter) at 37,000 rpm for 170 min at 4° C. The fractions were obtained with a Biological LP System (Bio-Rad). 14 fractions were collected from each sample, with each fraction having a final volume of 600 μl. From the initial 14 fractions, 4 final samples were obtained (by pulling together some of them): 40S, 60S, 80S, and polysomes.

qPCR

RNA was reverse transcribed using the High-Capacity complementary DNA Reverse Transcription Kit (Thermo Fisher Scientific) on a Veriti 96-well thermal cycler (Thermo Fisher Scientific). Gene expression was measured by qPCR on a QuantStudio 5 (Thermo Fisher Scientific) and normalized using 28S and 18S as reference genes (for polysome profiling experiments) or the average of HPRT, TBP, and UBC. The following primer sequences were used for LINC00941:

LINC00941 FW:
(SEQ ID NO. 1)
GCC AGC TGA CAA CTT GAT TGG GTT;
and,
LINC00941 RW:
(SEQ ID NO. 2)
GGC GCT TCA AAC CTG AAG GA

### Co-Culture With PBMCs

MM099 cells were plated in a 6 well (150 000 cells per well). 24 h after seeding, knock-down (KD) of LINC00941 were performed, at the usual concentration of 25 nM. The same day of the KD, 1.5 million peripheral blood mononuclear cells (PBMCs) (HLA matched) were grown in 1.5 ml of RPMI, complemented with complemented with CD28 (5 ug/ul), CD3 (3 ug/ul) and IL2 (200 ng in total). 48 h after transfection, the MM099 were reseeded in a 96 well plate (1500 cell per well) in fresh medium and co-cultured with PBMCs (7500 cell per well) labelled with Nuclight Rapid Red Dye. PBMCs were also cultured in the medium derived from transfected cells at 48 h from transfection in the absence of melanoma cells. The ratio melanoma cells vs PBMCs was 1:5. Differences in cell numbers were calculated with incucyte over 48 h.

### Treatment of an PDX Melanoma Model Resistant to Immune Checkpoint Blockade

The cutaneous melanoma PDX models are part of the Trace collection (https://www.uzleuven-kuleuven.be/lki/trace/trace-leuven-pdx-platform) and were established using metastatic melanoma lesions derived from patients undergoing surgery as part of standard treatment at UZ Leuven. Tumours were engrafted into 23 weeks old NOD/Shi-scid/IL-2Rγnull mice, humanized with CD34⁺ cells engrafted at the age of 5 weeks with CD34⁺ hematopoietic stem cells from 3 different donors. When the tumor reached the size of 100 mm³, treatment with ASO (15 mg/kg sub cutaneous every second day) and/or nivolumab (10 mg/kg twice a week i.p.) was initiated. The study was terminated when the mice reached 1500 mm³.

### Reagents for LINC00941 Knock-Down

A pool of the 2 siRNAs at the final concentration of 25 nM was transfected using lipofectamine 2000. Knock-down efficiency was measured by RT-qPCR 48 h after transfection.

For ASO knock-down 15 nM concentration overlapping the sequence recognized by siRNA #3 was used. Knock-down efficiency was measured by RT-qPCR 24 h after transfection.

Sequences targeted by siRNAs and ASO on LINC00941:

siRNA #1:
(SEQ ID NO. 3)
GAGACAGUUGAUAGCCAAA
siRNA #3 and ASO:
(SEQ ID NO. 4)
AAGCAUGCACCACUACACUCA

The ASO sequence used was a Gapmer of nucleic acid sequence: 5′-TGAGT GTAGTGGTGCA TGCTT-3′ (SEQ ID NO. 5) wherein 5 residues at the 5′ and 5 residues at the 3′ ends (in bold font) are locked nucleic acids and wherein the central part (italicized) is DNA. Phosphodiester bonds are replaced by phosphothioate linkage as in the entire sequence. The modified sequence is SEQ ID NO. 6.

### Results and Conclusions

Translation rewiring through activation of the Integrated Stress Response (ISR) allows normal cells to adapt to stress and cancer cells to evade therapy. The ISR consists in a global reduction of CAP-dependent translation, accompanied by activation of the transcription factor ATF4. In melanoma, this adaptive pathway was shown to confer resistance to MAPK inhibitors and to have pleiotropic effects on the regulation of the immune responses. Despite having a low coding potential, a large portion of long non-coding RNA (IncRNAs) is consistently associated with ribosomes. Our data, supports a role for these transcripts as regulators of translation and ISR-dependent translation rewiring. Considering the increasingly recognized role of the ISR in targeted therapy (TT) and immune therapy (IT) resistance, the identification of cancer cell-specific IncRNAs regulating this stress response pathway may lead to the development of innovative therapeutic strategies capable of overcoming therapy resistance.

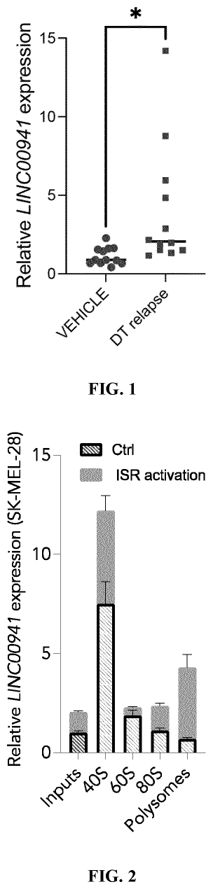

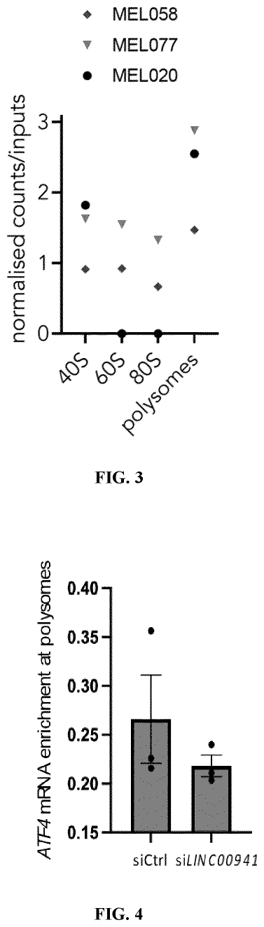

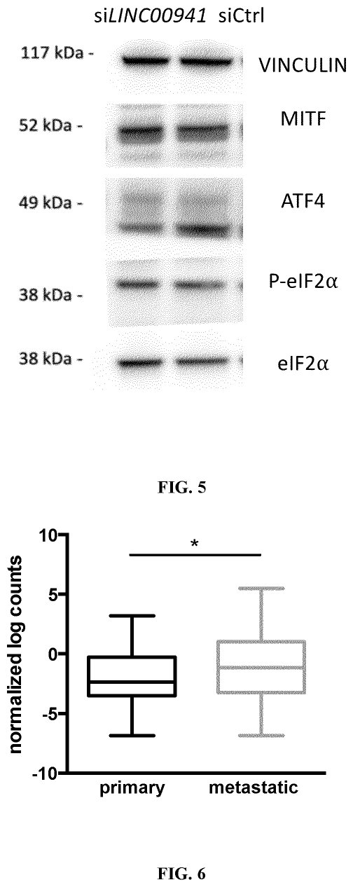

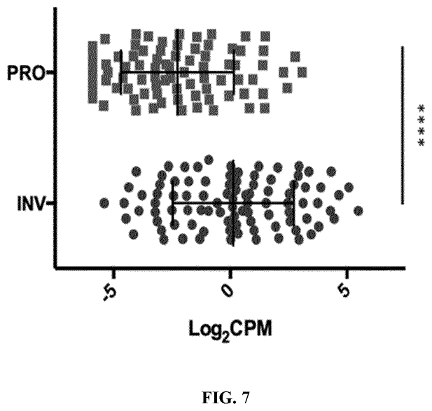

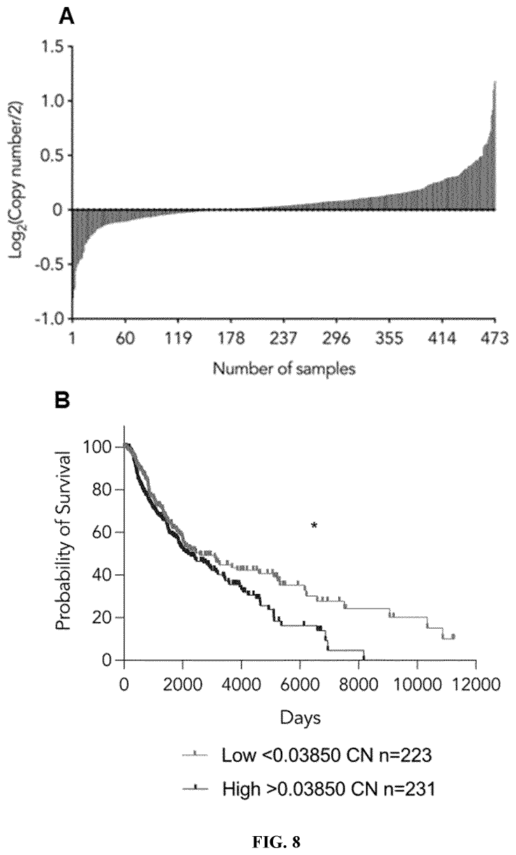

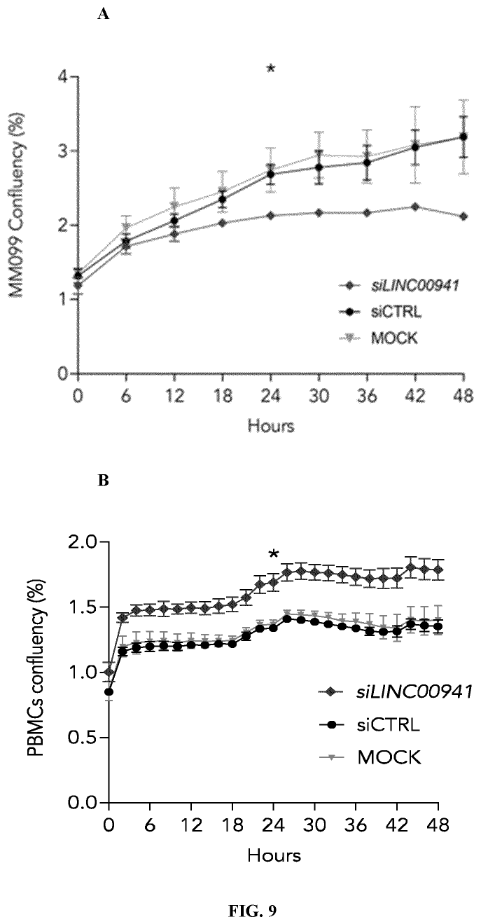

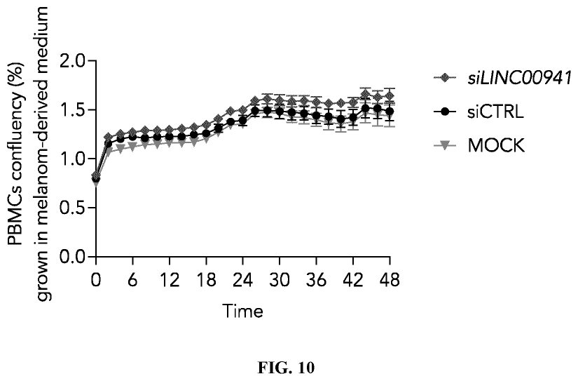

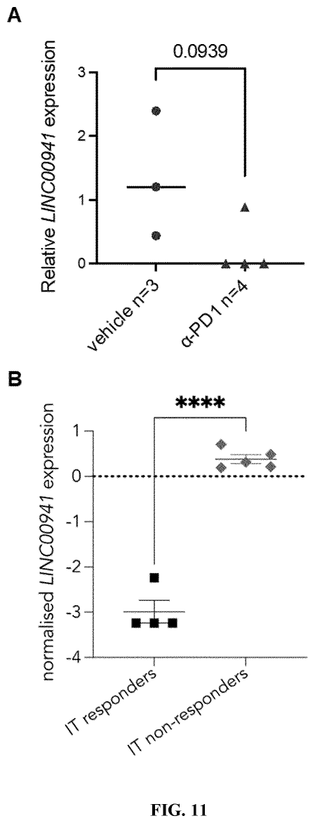

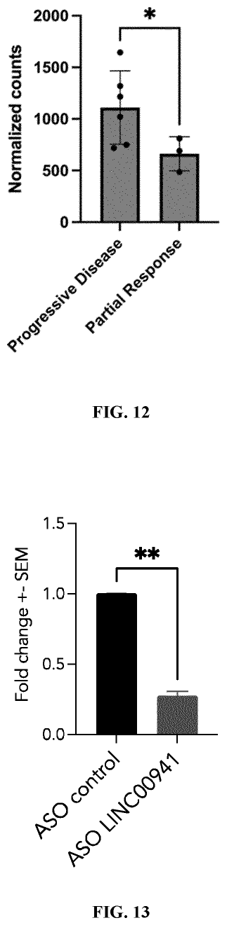

In the present application, LINC00941 was unexpectedly identified because of its upregulation in melanoma Patient-Derived Xenograft (PDX) upon acquisition of resistance to targeted therapy (FIG. 1). It is demonstrated that LINC00941 binds to the 40S ribosomal subunit in melanoma lines but it is enriched at polysomes upon induction of drug-tolerance in cell lines (FIG. 2) and in therapy-resistant PDXs (FIG. 3) to regulate translation of ATF4 (FIG. 4 and FIG. 5), a major driver of translation inhibition during ISR. LINC00941 is not expressed in normal tissues, but in TCGA melanoma cohort, it is elevated in metastatic samples having a higher invasive/mesenchymal signature (FIG. 6 and FIG. 7). Additionally, LINC00941 locus is amplified in about 60% of the patients (FIG. 8 panel A) and the copy number anticorrelates with patients' survival (FIG. 8 panel B). Survey of translated genes in LINC00941 KD cells by polysome profiling and RNAseq, suggests a role for this IncRNA in immune evasion. Accordingly, LINC00941 KD unleashes T-cell killing mediated by cell-cell contact (FIG. 9 and FIG. 10) in co-culture experiments. In keeping with this, LINC00941 expression is downregulated in response to anti-PD1 in vivo in humanized PDXs responding to anti-PD1 treatment (FIG. 11 panel A) and LINC00941 expression anticorrelates with responses to checkpoint inhibition in humanized PDXs (FIG. 11 panel B). Furthermore, this is also confirmed in a patient cohort of partial responders (FIG. 12). A good LINC00941 KD is also obtained by using an ASO (FIG. 13).

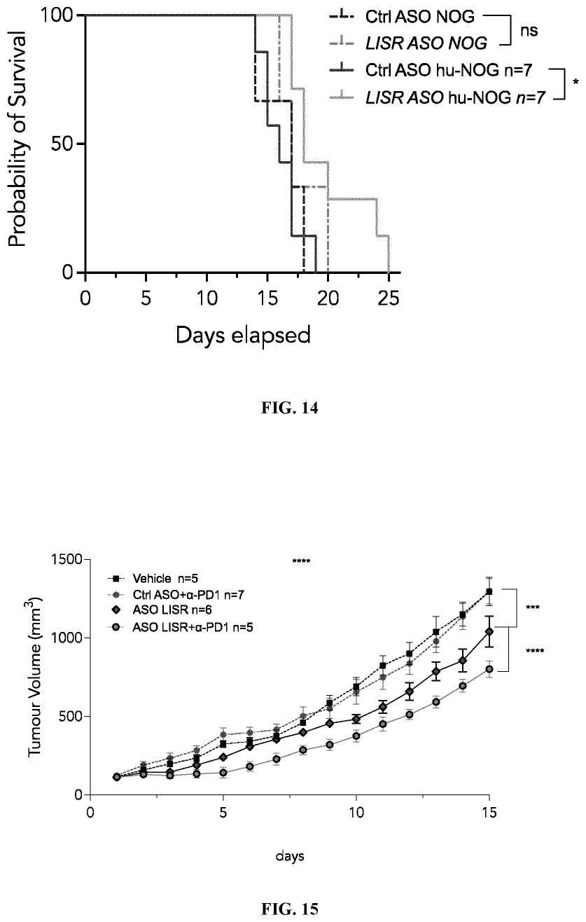

Systemic inhibition of LINC00941 does not affect growth of a melanoma PDX model in immunocompromised mice (NOG) but it significantly increases overall survival when the 10 same model, not responding to immune checkpoint inhibitor, is implanted in immunocompetent mice (hu-NOG) (FIG. 14). Additionally, LINC00941 inhibition resensitizes the model to immune checkpoint blockade (FIG. 15).

### REFERENCES

Hugo et al., Genomic and Transcriptomic Features of Response to Anti-PD-1 Therapy in Metastatic Melanoma, Cell. 2016 Mar. 24;165(1):35-44.

Larkin et al., Five-Year Survival with Combined Nivolumab and Ipilimumab in Advanced Melanoma, N Engl J Med. 2019 Oct. 17;381(16):1535-1546.

Vendramin et al., Activation of the integrated stress response confers vulnerability to mitoribosome-targeting antibiotics in melanoma, J Exp Med. 2021 Sep. 6;218(9):e20210571).

1. An inhibitor of LINC00941 expression for use in the treatment of melanoma in a subject.

2. The inhibitor of LINC00941 expression for use according to claim 1, wherein said inhibitor of LINC00941 expression is selected from the group consisting of silencing RNA (siRNA) and antisense oligonucleotide (ASO).

3. The inhibitor of LINC00941 expression for use according to claim 1 or 2, for use in combination with an immune checkpoint inhibitor and/or a therapeutic agent for targeted therapy.

4. The inhibitor of LINC00941 expression for use according to any one of claims 1 to 3, wherein said subject has, or is at risk of having, melanoma resistant to an immune checkpoint inhibitor and/or to a therapeutic agent for targeted therapy.

5. The inhibitor of LINC00941 expression for use according to any one of claims 1 to 4, wherein said melanoma is, or is at risk of being, a melanoma resistant to an immune checkpoint inhibitor and/or to a therapeutic agent for targeted therapy.

6. The inhibitor of LINC00941 expression for use according to any one of claims 3 to 5, wherein said immune checkpoint inhibitor is selected from the group consisting of PD-1 inhibitors, CTLA-4 inhibitors, PD-L1 inhibitors and LAG3 inhibitors.

7. The inhibitor of LINC00941 expression for use according to any one of claims 3 to 6, wherein said therapeutic agent for targeted therapy is selected from the group consisting of B-Raf inhibitors and MEK inhibitors.

8. The inhibitor of LINC00941 expression for use according to any one of claims 1 to 7, wherein said melanoma is invasive melanoma and/or metastatic melanoma.

9. The inhibitor of LINC00941 expression for use according to any one of claims 1 to 8, wherein said inhibitor of LINC00941 expression is an ASO targeting LINC00941.

10. The inhibitor of LINC00941 expression for use according to any one of claims 1 to 9, wherein said inhibitor of LINC00941 expression is an ASO inducing degradation of LINC00941, preferably inducing RNAse H-mediated degradation of LINC00941.

11. The inhibitor of LINC00941 expression for use according to any one of claims 1 to 10, wherein said inhibitor of LINC00941 expression is an ASO having an overall nucleotide sequence length of at least 10 nucleotides, preferably an ASO having an overall nucleotide sequence length of at least 12, 13, 14, 15, 16, 17, 18, 19 or 20 nucleotides, more preferably an ASO having an overall nucleotide sequence length of at least 21 nucleotides.

12. The inhibitor of LINC00941 expression for use according to any one of claims 1 to 11, wherein said inhibitor of LINC00941 expression is an ASO comprising a contiguous nucleotide sequence that is at least 90%, 92%, 94%, 96%, 97%, 98% 99% or preferably 100%, complementary to LINC00941 and that is of length of nucleotide sequence ranging from 10 to 50 nucleotides, preferably ranging from 10 to 40, 10 to 39, 10 to 38, 10 to 37, 10 to 37, 10 to 36, 10 to 35, 10 to 34, 10 to 33, 10 to 31 or 10 to 30 nucleotides, more preferably ranging from 11 to 29, 12, to 28, 13 to 27, 14 to 26 or 15 to 25 nucleotides.

13. The inhibitor of LINC00941 expression for use according to any one of claims 1 to 12, wherein said inhibitor of LINC00941 expression is an ASO comprising modified nucleotides selected from the list comprising: 2′—O—Me, 2′—F, MOE, LNA or a combination thereof.

14. The inhibitor of LINC00941 expression for use according to any one of claims 1 to 13, wherein said inhibitor of LINC00941 expression is a gapmer.

15. A method for the treatment and/or prevention of melanoma in a subject in need thereof, said method comprising administering to said subject an inhibitor of LINC00941 expression.
